(19) United States
(12) Patent Application Publication
(10) Pub. No.: US 2012/0183594 A1
(43) Pub. Date: Jul. 19, 2012

(54) Method of increasing photosynthesis and reducing ozone

(75) Inventors: David Michael Glenn, Shepherdstown, WV (US); Peter S. Barrows, Washington Crossing, PA (US); Kurt C. Volker, Yakima, WA (US); Christopher G. Hayden, Alexandria, VA (US)

(73) Assignees: The United States of America as represented by the Secretary of Agriculture (Washington, DC); Tessenderlo Kerley, Inc., Phoenix, AZ (US)

(21) Appl. No.: 13/200,824

(22) Filed: Oct. 3, 2011

Related U.S. Application Data
(60) Provisional application No. 61/344,770, filed on Oct. 1, 2010.

(51) Int. Cl. A61K 9/70; A61K 35/74; A61K 35/66; A61K 9/16

(52) U.S. Cl. 424/443; 424/490; 424/93.4; 424/93.1; 424/93.43; 424/93.46

(57) ABSTRACT

A method of protecting plants from ozone by applying to the photosynthetically active portions of said plants a particle film containing particles, an effective amount of a volumizing and two or more of nitrogen-rich carbonaceous materials which destroy ozone, microbial fertilizer which promotes microbial growth in the particle film, and ozone-reactable carbonaceous materials coated on the particles.

The present application claims priority to pending U.S. Provisional Application 61/344,770 filed on Oct. 1, 2010. For the United States application, this application also claims priority to application Ser. No. 11/463,883 filed Aug. 10, 2006, pending, to U.S. provisional Application No. 60/595,862 filed Aug. 11, 2005; and to application Ser. No. 12/805,583 filed Aug. 10, 2010, and to application Ser. No. 11/464,023 filed Aug. 10, 2006, now U.S. Pat. No. 7,781,375, and to U.S. provisional Application No. 60/595,858 filed Aug. 11, 2005; and to application Ser. No. 11/380,639 filed Apr. 27, 2006, and to U.S. provisional Application No. 60/594,918 filed May 18, 2005, the entire contents of which are incorporated herein by reference thereto for all lawful purposes.

### FIELD OF THE INVENTION

The present composition is capable of forming a persistent particle film on a plant surface, said particle film capable of greatly reducing ozone damage to plants and also reducing the quantity of ozone in the air around the plant.

### BACKGROUND

Background ozone levels in unpolluted air can be anywhere from 20-50 ppb. Polluted regions can have ozone levels peaking as high as 400 ppb. Ozone is known to adversely affect photosynthesis. Physiological effects of ozone exposure include reduced photosynthesis, increased turnover of antioxidant systems, increased dark respiration, reduced carbon transport to roots, and reduced forage quality of C4 grasses. Response to ozone varies among species. Various studies have concluded that elevated levels of ozone result in a 50% reduction in photosynthesis for crops such as clover and wheat, but only a 10% reduction for white pine.

Even background ozone levels have great effect on the photosynthesis of some plant species. Various models suggest that an ozone dose of 20 ppb results in a photosynthesis reduction of 7% for conifers, 36% for hardwoods, and 73% for crops. Reduced photosynthesis results in decreased growth rates, which are often measured as either volume or biomass. This corresponds to a growth reduction of 3% for conifers, 13% for hardwoods, and 30% for crops. The Southern Oxidant Study concluded that ozone had led to a 1-25% growth reduction in eastern U.S. forests. The Southern Appalachian Mountains Initiative concluded that black cherry and yellow poplar were the most sensitive to ozone, while red maple, loblolly pine, and northern red oak were more tolerant.

Many studies have detailed the reduction of crop yield and photosynthesis by exposure to ozone. The National Crop Loss Assessment Network program results indicate a reduced annual soybean yield of 10% and a reduced cotton yield of 12% for seasonal mean ozone levels greater than 50 ppb. Corn is less sensitive, but a 0.3% to 0.9% increase in corn and soybean yield could be obtained in the eastern USA with a 20 ppb summer ozone exposure reduction.

Ozone uptake is a function of both ambient ozone levels and stomatal conductance. Ozone affects vegetation by direct cellular damage once it enters the leaf through the stomates. Gaseous O3 diffuses from the atmosphere, through the stomata, and dissolves in water surrounding the cells before entering the cells themselves. A secondary response to ozone is a reduction in stomatal conductance, as the stomata close in response to increased internal CO2 that occurs because of the reduced photosynthetic activity caused by the ozone. See B. S. Felzer et al., C. R. Geoscience 339 (2007), published by Elsevier Masson SAS.

Use of particle films in agriculture is known. Several commercial brands, for example Surround® and PurShade®, are engineered to provide a highly reflective surface which can diffuse light and reduce canopy temperature, thereby in certain instances increasing photosynthesis. Use of organic sprays are also know. Orchid and rose growers use alfalfa tea as a foliar spray. Alfalfa meal is used as a fertilizer, and can contain ˜4-5.5% nitrogen, 0.75 to 3% potassium, 1-2% calcium, 0.3-1% magnesium, 0.2-0.5% sulfur, and trace metals including ˜100 ppm manganese, 3100 ppm iron, 50 ppm boron, 10 ppm copper, and 30 ppm zinc.

### SUMMARY OF THE INVENTION

In one embodiment the present invention is a method of protecting plants from ozone comprising: applying to the plants a particle film containing

A) between about 50% and 99.4%, for example between 70% and 90% by weight of particles selected from the group consisting of mineral particles, polymeric particles and/or fibers, cellulosic powder and/or fibers, and charcoaled (activated) carbon particles;

B) at least one volumizing agent in an effective amount, for example between 0.35% and 15% by weight, typically between 0.35% and 5% by weight and in one embodiment being selected from the group consisting of: (i) modified cellulose (ii) a polyacrylate or polymethacrylate; (iii) a gum, and (iv) a polyacrylamide, (v) nitrogen-containing polyamine polymers such as polydiallyldimethylammonium chloride or polyaspartic acid, and vi) a high average molecular weight polyvinyl alcohol of molecular weight greater than 85000, preferably between 140000 and 240000, e.g., a 4 weight % solution showing a viscosity in water of 25 to 50 cp,;

C) two or more of:
c1: between 0.1% and 25% by weight, for example between 5% and 20% of active nitrogen-rich carbonaceous materials which destroy ozone, said materials being immobilized in the particle film and in preferred embodiments comprising one or more of polyamines, poly-amino acid derivatives;
c2: between 0.1% and 25% by weight, for example between 5% and 15% by weight of materials which promote microbial growth (microbial fertilizer) on and in the particle film and selected from slow release fertilizer particles (slow release meaning particles holding more than about half of original fertilizer during slurrying and spraying, until after particle film dries) and microflora nutrients including primarily sources of C and N; and
c3: between 0.1% and 25% by weight for example between 5% and 20% by weight of active carbonaceous materials coated on the particles, said carbonaceous materials comprising ozone-reactable carbon sources, for example organic teas, such as alfalfa teas, compost teas, fermented organic solutions, and the like, and

D) optionally one or more of: 0.01% to 10%, for example 0.1% to 5%, of beneficial bacteria or microflora; fatty acid esters of ascorbic acid; 0.1% to 10% of a spreader/surfactant that causes the film to spread across a plant leaf surface; effective amounts of biologically active agents which can ameliorate oxidative damage, i.e., ascorbic acid, azealic acid, salicylic acid, kojic acid, and the like, for example present in amounts from about 1 ppm to about 100 ppm; and 0.01% to 20% of a phthalocyanine dye, for example pigment green 7; said particle film having a dry weight of between 25 and 5000 micrograms per square centimeter.

In preferred compositions there is at least 0.35%, preferably between 0.35% and 5% by weight of a volumizing agent, between 5% and 20% of active nitrogen-rich carbonaceous materials which destroy ozone, between 0.1% and 25% by weight, for example between 5% and 15% by weight of materials which promote microbial growth (microbial fertilizer) on and in the particle film; and 5% and 20% by weight of ozone-reactable carbonaceous materials carbon sources, wherein the minimum amount of carbonaceous material excluding the particles is at least 15% by weight, preferably at least 20% by weight, for example between 20% to 60%, more typically between 20% and 25%. Use of such high organic loadings can become even more long-lasting is some amount of the particle film, say at least 10% by weight, say between 20 and 70% by weight of the particles are cellulosic particles. In one embodiment at least 15% of the particles are mineral particles.

In another embodiment the present composition is a persistent particle film on a plant surface, said particle film capable of greatly reducing ozone damage to plants and also reducing the quantity of ozone in the air around the plant, said particle film comprising:
A) between about 50% and 99.4%, for example between 70% and 90% by weight of particles selected from the group consisting of calcium carbonates, kaolinites, attapulgite, bentonites, calcined kaolinite, polymeric particles and/or fibers, cellulosic powder and/or fibers, and activated carbon particles.
B) at least one volumizing agent in an amount between 0.35% and 15% by weight, typically between 0.35% and 5% by weight and in one embodiment being selected from the group consisting of: (i) modified cellulose (ii) a polyacrylate or polymethacrylate; (iii) a gum, and (iv) a polyacrylamide, (v) nitrogen-containing polyamine polymers such as polydiallyldimethylammonium chloride, and vi) a high average molecular weight polyvinyl alcohol of molecular weight greater than 85000, preferably between 140000 and 240000, e.g., a 4 weight % solution showing a viscosity in water of 25 to 50 cp,;
C) one or more of:
D) c1: between 0.1% and 25% by weight, for example between 0.1 and 20% of active carbonaceous materials which destroy ozone, said materials being immobilized in the particle film and in preferred embodiments comprising one or more of polyamines, poly-amino acid derivatives, derivatives (e.g, fatty acid esters) of ascorbic acid or erythorbic acid, and/or mixtures thereof;
   c2: between 0.1% and 25% by weight, for example between 0.1% and 15% by weight of materials which promote microbial growth (microbial fertilizer) on and in the particle film and selected from slow release fertilizer particles and microflora nutrients including especially sources of C and N, for example ammonium sulfate, phosphates, ureas, phosphonates, amino acids, and (poly)aspartic acid; and
   c3: between 0.1% and 25% by weight of active carbonaceous materials coated on the particles, said carbonaceous materials comprising ozone-reactable carbon sources, for example organic teas, such as alfalfa teas, compost teas, fermented organic solutions, and the like,
E) optionally one or more of: 0.01% to 10% beneficial bacteria or microflora; 0.1% to 10% of a spreader/surfactant that causes the film to spread across a plant leaf surface; effective amounts of biologically active agents which can ameliorate oxidative damage, i.e., azealic acid, salicylic acid, kojic acid, and the like, for example present in amounts from about 1 ppm to about 100 ppm; and 0.01% to 20% of a phthalocyanine dye, for example pigment green 7.

All percentages unless otherwise specified are weight percent of the dried particle film, applied for example as a slurry to foliage of a tree or crop. Particularly useful are treatments on certain crops, e.g., watermelon, lettuce, cotton, grape, and tomato.

Particle films are known to protect plants from sunburn. Typical prior art films are made substantially of white mineral particles, primarily kaolins and calcites. Generally, particle films are sprayed on plants in the form of a formulated slurry, and the slurry may further comprise a small amount of surfactants, fertilizers, and the like. Particle films having some surfactants would be expected to degrade some ozone. The amount of ozone degradation from such prior art particle films would not be significant, and the ozone degradation may not result in increased photosynthesis. The inventive concept here is to provide a particle film that provides to a treated plant a significant and substantial protection from ozone. Significant protection can be, for example, amelioration of ozone-related photosynthesis damage by an amount equivalent to a reduction of at least 10 ppb of ozone. This effect is separate from increased photosynthesis resulting from the bright reflective and optimally diffusive effects of particle films, which can by themselves increase photosynthesis. For example the particle film of the current invention may ameliorate ozone damage by an amount at least equivalent to what would be demonstrated by the plant if exposed to reduced ambient ozone exposure, say by 20, or by 40 ppb, or by 60 ppb, of ozone. Each species has different responses to ozone—some species are resistant to ozone damage, some species are susceptible to ozone damage, and some species are resistant to ozone up to certain levels. And the maximum amount of ambient ozone on a sunny day can vary from 40 ppb to over 200 ppb, depending on location, temperature, and the like, and 200 ppb ozone significantly impair plant health and photosynthesis. Additionally, air flow by treated plants can result in an actual reduction ozone in the ambient air, though the effect for single trees is only a few ppb decrease in ambient ozone even under conditions of substantially no wind.

### LIST OF FIGURES

The following is a brief description of the Figures:

FIG. 1 is a graph showing results of ozone degradation tests with air flowing by a kaolin/organic particle film.

FIG. 2 is a graph showing results of ozone degradation tests with air flowing by a kaolin/organic particle film.

FIG. 3 is a graph showing results of ozone degradation tests with air flowing through a kaolin/organic particle film.

FIG. 4 is a graph showing results of ozone degradation tests with air flowing through a kaolin/organic particle mass.

FIG. 5 is a photograph of ozone flow test chambers with samples therein.

FIG. 6 is a graph showing results of ozone degradation full tree field tests with ambient air flowing through a tree canopy that was treated with a kaolin particle film.

FIG. 7 is a graph showing results of photosynthesis rates of plants in field tests with high levels of ozone, where plants were treated with a particle film of kaolin and alfalfa dust and alfalfa tea.

### DETAILED DESCRIPTION OF THE INVENTION

The present composition is capable of forming a persistent particle film on a plant surface, said particle film capable of greatly reducing ozone damage to plants and also reducing the quantity of ozone in the air around the plant. Particle films are known in the art. Most commercially available particle films for use on plants are based on calcite or kaolin, often contain about 0.5% of dispersants, and are used to treat sunburn and heat stress. Certain highly refined commercially available particle films, such as the Surround® calcined kaolin product and the Purshade® calcium carbonate product, each available from NovaSource, Tessenderlo Group, are known to increase photosynthesis and carbon assimilation by treated plants, and to reduce arthropod infestations. Some particle films, e.g., Eclipse™, purport to be a calcium and boron supplement for the treated plants.

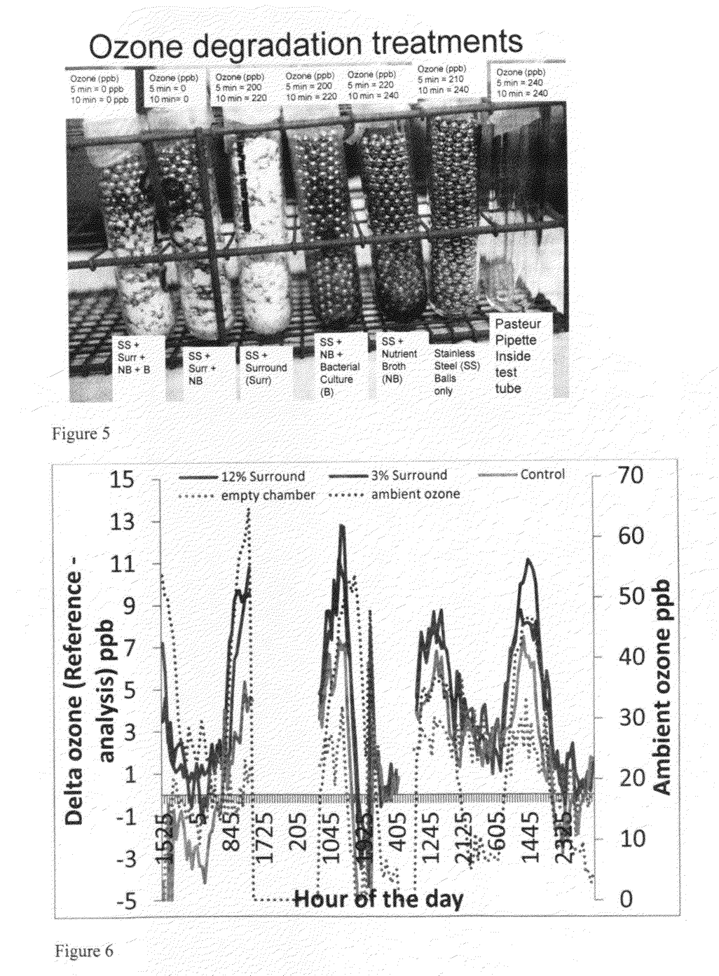

Ozone damage has become a very significant problem with a number of plant species. Ozone (O₃) is a metastable molecule, in that it reacts with certain moieties, for example hydroxyl groups (OH) in an organic molecule, to revert to oxygen and water. These hydroxyl groups are the source of hydrogen bonding in organic molecules that gives them their functional 3D structure. It is difficult to perform quantitative studies on the effects of various substrates on ozone, as ozone reacts with so many materials. In initial screening tests, an air stream of 5 and 10 ml/sec containing about 240 ppb ozone was passed through chambers filled with ˜⅛ inch steel beads. The chambers were small so residence time of gas in the chamber was on the order of one second. The test chambers and results are shown in FIG. 5. Air having 240 ppb ozone passing through the chamber containing only steel balls contained ˜210 ppb ozone at the exit. All the tests described here were performed on essentially dry substrates. The broth introduced in certain tests was substantially dry when run in the ozone flow chambers, and bacteria introduced in certain tests was substantially dry and inactive when in the ozone flow chambers. If the steel balls were soaked in a certain amount of nutrient broth and dried so that the broth deposited on the steel balls, air having 240 ppb ozone passing through the chamber contained ˜210 ppb ozone at the exit. The same result occurred with steel balls soaked with a certain amount of nutrient broth and bacteria-air having 240 ppb ozone passing through the chamber contained ˜210 ppb ozone at the exit. Surprisingly, the same result occurred with steel balls coated with a certain amount of Surround® brand calcined kaolin particle film. Again the ozone level at the chamber exit was 200 to 220 ppb. Note Surround® contains about 95% calcined kaolin, ˜0.5% of organic surfactant/dispersant/volumizing agents, and some hydrous kaolin.

Useful volumizing agents are disclosed in US application 20100304974, which is incorporated by reference thereto. Volumization agents, such as animal glue, water-soluble polymers including polyacrylamide (PAM), certain polyamines (epichlorohydrin-dimethylamine); or polyacrylate materials, polydiallyldimethylammonium chloride (polyDADMAC) and epichlorohydrin-dimethylamine (Epi-DMA). Polyacrylates have the repeating unit —[CH₂—CR(CO₂R)]ₙ— wherein each R is independently a hydrogen, or alkoxy or alkyl group containing 1 to about 4 carbon atoms, and n is from about 250 to about 10,000. In another embodiment, each R is independently a hydrogen or methyl group and n is from about 500 to about 5,000 Daltons. The phrase “high molecular weight”, used in connection with high molecular weight polyacrylates, and high molecular weight polyacrylamides, means having an average molecular weight of at least about 25,000 Daltons, and typically about 25,000 to about 1,500,000 Daltons. In another embodiment, high molecular weight means having an average molecular weight of at least about 50,000 Daltons, and typically about 50,000 to about 1,000,000 Daltons. In yet another embodiment, high molecular weight means having an average molecular weight of at least about 75,000, and typically at least about 75,000 Daltons to about 500,000 Daltons. Examples include polymethylacrylate, polyethylacrylate, polyacrylic acid, polymethylmethacrylate, polyethylmethacrylate, poly (2-hydroxyethyl methacrylate), and the like.

Clearly, calcined kaolin films alone, even films having a commercially reasonable amount of surfactant/dispersant/volumizing agents, have little effect on ozone. When the amount of nutrient broth was mixed with the Surround® and coated on the steel balls, the ozone concentration at the exit dropped to essentially zero. The same result not surprisingly was seen with a Surround/broth/bacteria coating on the steel balls. We believe the effect is related to the high surface area and/or to the three-dimensional structure of the Surround® film. Surround contains ˜95% calcined clay, and over-laying particles of calcined kaolin do not lay flat like particles of hydrous kaolin.

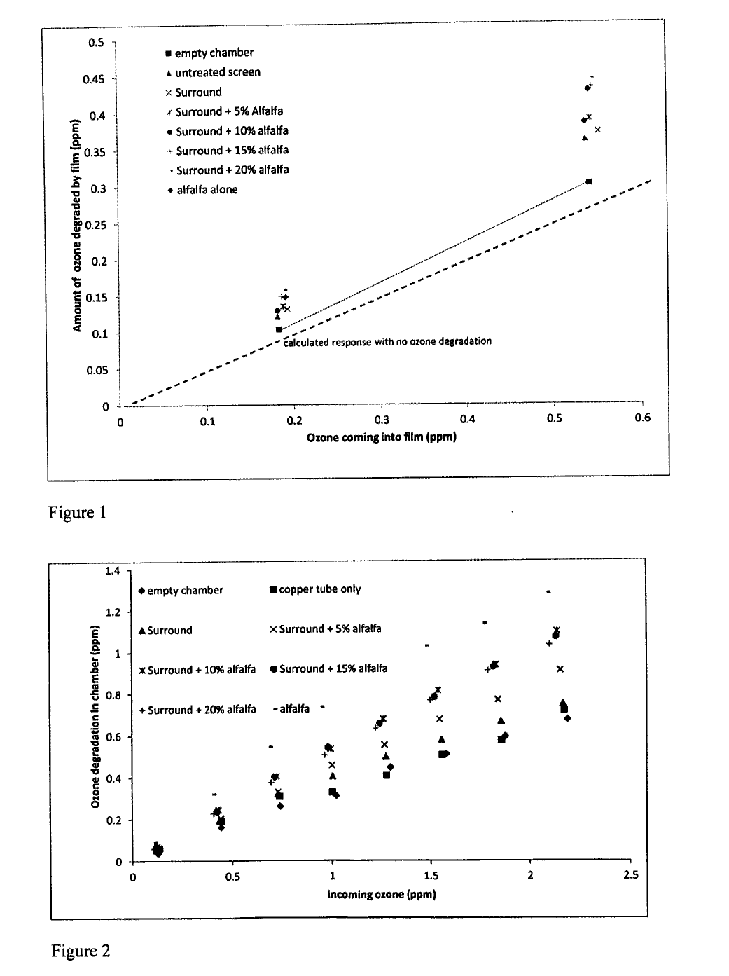

In subsequent tests, Surround alone was observed to have a small ozone degradation factor, but the ozone degradation increased dramatically with addition of a small amount of organic material which would coat clay particles. We ran tests where air/ozone was passed through a chambers containing a tube, a tube coated with a dry film of Surround®, and the tube coated with dry films having Screen/Surround/alfalfa tea, where the film comprised 5% to 25% alfalfa tea by weight. Simply passing ozone through the chamber and plumbing reduced ozone levels considerably. For ozone levels of 190 ppb and 540 ppb, the presence of Surround® reduced ozone levels by about of about 7 ppb. For ozone levels of 190 ppb and 540 ppb, a 10% tea/Surround® film reduced ozone by 11 ppb and by 20 ppb, while a 15% tea/Surround® film reduced ozone by 22 ppb and 65 ppb, respectively. Clearly, while the presence of Surround® had a small degrading effect on ozone, the presence of relatively small amounts of readily available organic material greatly increased ozone degradation. Additionally, changes in carbon dioxide content of outlet gas suggest the ozone was reacting with and oxidizing the organics. A number of these tests were run, and representative results can be seen in FIGS. 1 and 2.

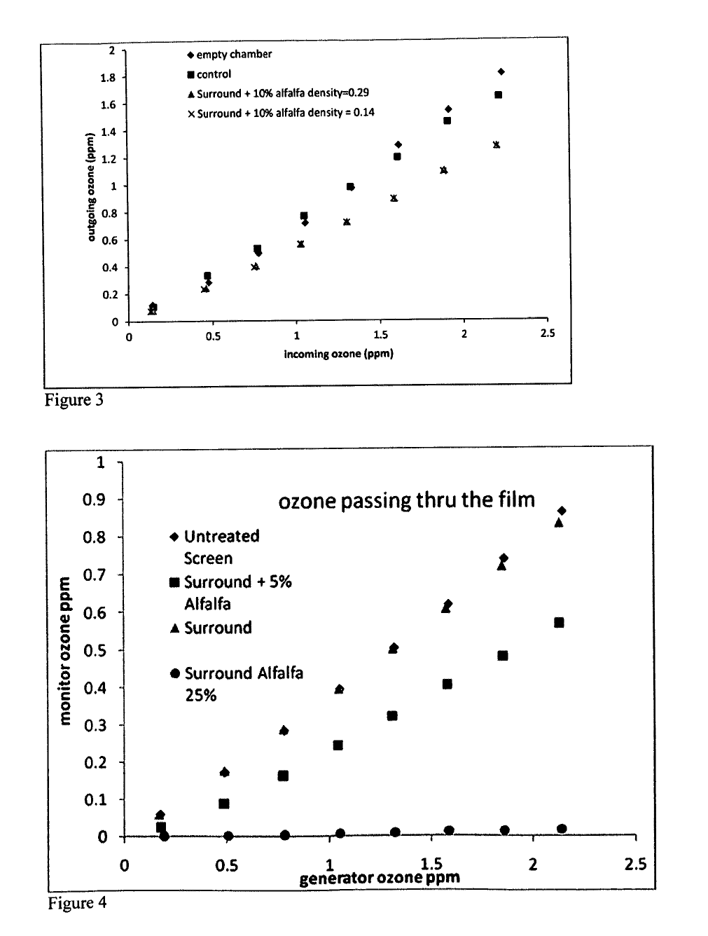

Surround® brand particle films are intended to be deposited on plants and to form a film on drying, and the Surround® contains altered clays and organic additives which promote a three dimensional structure. In the laboratory, which involves fast flow conditions, tests were made on different densities of particle films. Data is shown in FIG. 3. It seems impossible to measure the effect of the particle film on ozone degradation in the dimension from the outside of the PF (air) to the inside (stomata-side). Fast flow laboratory experiments suggest ozone degradation by organics is a 2 dimensional effect, ie. simply the surface area in contact with the moving air. There is no measurable degradation occurring as the air/ozone move into and through the <1 mm particle film. FIG. 3 shows no effect of ozone moving over a very porous particle film versus very compacted particle film, the idea being a less dense film has greater porosity and more diffusion into it. No such results were found. When ozone-containing air was forced through particle films containing various loadings of organic material, however, the results as shown in FIG. 4 clearly showed the organics contribution to deteriorating ozone. The experiments described above were fast flow experiments that do not necessarily reflect conditions on a leaf, where mass transport and diffusion might be much slower than in the dynamic flow laboratory conditions.

The conclusion was that Surround® films caused a modest degradation in ambient ozone, but the degradation increased significantly when organics coated the Surround® film. Most particle films would be expected to contribute slightly to ozone degradation, both from reactive sites and also by ozone reacting with any surfactants used in the particle film. However, two problems were observed when using simple organic material mixed with a particle film. First, the ozone degradation effects of a particle film coated with organics seems to be relatively short-lived, presumably as readily available ozone-reactive organics are consumed. Second, particle films containing fermented organics such as alfalfa tea contribute to disease growth on infected plants.

Additionally, particle films where particles were substantially covered with organics have also been tested. See, e.g., co-owned application 20030077309 titled Pesticide Delivery System where particles used in a particle film were made hydrophobic by addition of fatty acids such as stearic acid and stearate salts. We have previously observed that particle films containing hydrophobic particles, e.g., kaolin particles treated with fatty acids, can under some conditions trap water and therefore contribute to disease in infected plants.

Therefore, what is needed is a particle film wherein the particle film is largely hydrophilic, but wherein organic material at least partially coats a sufficient number of particles, and wherein said organic material is sufficiently reactive to ozone, so that the covered surfaces of the plant, crop, or tree are protected from the adverse effects of ambient ozone. To be useful the treatment should be long-lasting. If periodic re-treatment is expected, then a sufficient amount, say 5% to 25% by weight based on the weight of the particle film, of any organic, e.g., alfalfa tea, alfalfa dust, or extract of alfalfa, can effectively reduce a plants negative response to excessive levels of ambient ozone. To be effective, the amount of material should be sufficient to form a film, i.e., between 25 and 5000 micrograms, typically between 100 and 3000 micrograms, and usually between about 100 and 500 micrograms of particle film per square centimeter of treated plant surface. The important factors in particle films directed toward reducing ozone are permeability and the availability of a high surface area containing carbonaceous material that readily reacts with ozone. Therefore, lower use rates, e.g., 25 to 500 micrograms of particle film per square centimeter, for example 50 to 300 micrograms of particle film per square centimeter, can be useful providing a sufficient three dimensional film is created.

A primary ozone degrading agents in a particle film are nitrogen-rich carbonaceous materials which destroy ozone, where said nitrogen-rich carbonaceous materials means compounds that contain more than one nitrogen and that have at least one nitrogen per eight carbon atoms. This material is more resistant to degradation by ozone and byproducts are very useful nutrients for microflora. Another primary ozone degrading agents in a particle film are active carbonaceous materials which react with ozone, and which have more than 8 carbon atoms per nitrogen atom. Generally, ozone is reactable with organics containing C—O bonds, C—N bonds, N—O bonds, and OH groups. The more of these reactable bonds, often the quicker ozone neutralization. The third primary ozone degrading agents in a particle film are microflora and bacteria. These materials can advantageously be seeded onto the particle film, but even more importantly the microorganism can in the presence of moisture and microbial fertilizer regenerate.

Use of alfalfa as a source of carbon is not particularly critical. Alfalfa tea was used because the cost is relatively low. Any organic carbon source, such as, alfalfa powder, glucose, sucrose, corn starch, apple pumice, casein, or other inexpensive source, fixed to the architectural framework of the particle film surfaces, will suffice. But the carbon source is advantageously not readily soluble or it will be washed out of the particle film by rain, so more fixed carbon sources are more useful.

Alternatively, the particle film can be seeded with self-rejuvenating sources of organic material. If a particle film contains nutrients in a form to be available to beneficial bacteria, then colonies of beneficial bacteria can propagate on the films. Advantageously the bacteria fixes carbon, thereby replacing carbon which becomes deactivated by long term exposure to ozone. Note that by active carbon we are not talking about “activated carbon” particles, but rather carbon in hydrocarbons that are susceptible to ozone attack. Activated carbon, i.e., charcoaled coconut husks, for example, is known to absorb ozone. However, it is not practical to provide a sufficient number of activated charcoal particles or a surface layer of activated charcoal on clay platelets in a particle film. As used herein the “active carbon” refers to organic molecules that in a substantially dry form are readily react-able with ozone.

Bacterial growth can be facilitated by providing a source of carbon, a source of nitrogen such as amino acids, trace nutrients, and the like. Ozone-degraded organic material can provide nutrients, as can organic materials released by the plant itself. One caution, however, is that the nutrients may be used by non-helpful bacteria, e.g., by detrimental and disease-forming bacteria and molds. Therefore, if a particle film is to be seeded with nutrients intended to promote or sustain a bacterial colony within the particle film, then the particle film itself is advantageously seeded with one or more useful non-damaging bacteria. Such bacteria can include for example Actinovate™, a commercially available bacteria product is anti-mildew on foliage. Other useful bioorganisms include Streptomyces, Bacillus sp., bryophytes, and the like. Therefore the particle film becomes a vehicle that promotes enhanced growth of the normal microflora as well as beneficial microflora, e.g., beneficial bacteria and fungi used for pest management that would otherwise be applied alone, providing a refuge (UV protection) as well as nutrients for the microflora. The microflora in the particle film in turn supplies carbonaceous material that can react with ozone passing over and through the film.

While foliar fertilization is well known in the art, the fertilizer particles here are very slow microbial nutrients designed and intended for very slow release within the film so that the nutrients are substantially trapped in the particle film, thereby being useful to microbes growing in the film. The amount of such foliar fertilizers will typically be insignificantly small with respect to the plant—the fertilizers are intended for microbes in the particle film, and are not intended for the plant. This will require very small particles of fertilizers, in the range of 0.1 to 2 microns in diameter, bound to the particle film such that the fertilizers become slow release. Binding low levels of fertilizers, e.g., ammonium sulfate, in polymeric particles which react with and hold the fertilizers, or with very small slow release fertilizers, is envisioned. Advantageously, the particle film will additionally comprise materials which provide both a jumpstart to beneficial microbial populations as well as sources of carbon and nitrogen, for example compost-tea, alfalfa tea, alfalfate particles, and the like.

Alternatively or additionally, certain carbon sources that react with ozone but are particularly resistant to degradation can be fixated into the particle film, for example in amounts between 0.1% and 20%. These organic compounds tend to be at least somewhat polar and water-soluble. It may be useful to have a small fraction of particles in the particle film to be hydrophobic, and to add hydrophobic fatty acid moieties to the organic compounds, to more readily fixate certain otherwise water-soluble organic compounds. The compounds most useful are polyamines. Simple polyamines are useful, e.g., putrescine and the like, and degradation of the polyamines can provide a nitrogen source to beneficial biomass within the particle film. However, more stable polyamines such as polyaspartic acid, beneficially of mole weight greater than 1000, or poly-amino acids such as polyglutamic acid provides a number of carbon-oxygen and carbon-nitrogen bonds, and these polymers are not readily washed from a particle film by rain. These polymers can both provide a readily accessible ozone-neutralizing carbon source to the particle film, and as these polymers are degraded by ozone, the byproducts are excellent nutrients for microflora in the particle film.

Ascorbic acid is a well-known antioxidant and cellular reductant that plays a primary role in the response of plants to ozone, typically forming the first line of defense against ozone in the apoplastic space. Sensitivity to ozone is typically correlated with total ascorbic acid levels. For activity, ascorbic acid must be in the fully reduced state. Therefore, both the rate of ascorbic acid synthesis and recycling via dehydroascorbate are critical in the maintenance of a high ascorbic acid redox state. Such processes are not possible in a particle film, unless maintained by a microorganism. However, inclusion of ascorbic acid or derivatives thereof is highly beneficial, because foliar applications of ascorbic acid have been shown to reduce ozone damage in plants and because microorganisms in the particle film can obtain ascorbic acid from the particle film and become more resistant to damage/death caused by ozone. That is, ascorbic acid alone in the particle film slurry will benefit both the treated plant and the microflora in the particle film, though any ascorbic acid not fixated by the treated plant or by the particle film microflora will be quickly washed away by rain. To fix a source of ascorbic acid in the film, use of ascorbic acid derivatives is beneficial. Ascorbic acid derivatives include, but are not limited to esters, ethers, and salts of ascorbic acid. With respect to the esters, they may be selected from the group consisting of C₇ to C₂₀ fatty acid mono-, di-, tri-, or tetra-esters of ascorbic acid (or erythorbic acid). Nonlimiting examples are monoesters such as ascorbyl palmitate (i.e., L-ascorbyl 6-palmitate), ascorbyl laureate, ascorbyl myristate, ascorbyl stearate, and also di-esters such as ascorbyl dipalmitate and tri-esters such as ascorbyl tripalmitate. Salts useful in this invention include ascorbic acid 2-phosphate salts including ascorbic acid-2-phosphoric esters, ascorbic acid 2-sulfate salts, and ascorbic acid 2-phosphate salts.

Other antioxidants known in the art, e.g., N-acetyl-L-cysteine, can also be beneficially added to a particle film slurry. Such anti-oxidants are known to be beneficial to treated plants.

In one embodiment, the particle film contains: between about 80 and 99.4% by weight of particles selected from the group consisting of calcium carbonate, kaolinite, attapulgite, bentonite, and/or calcined kaolinite, (b) at least one volumizing agent in an amount between 0.35% and 5% by weight, for example being selected from the group consisting of: (i) modified cellulose selected from the group consisting of hydroxy ethyl cellulose, carboxymethyl cellulose, carboxyethyl cellulose, ethyl hydroxy ethyl cellulose, hydroxy propyl cellulose, hydroxy ethyl methyl cellulose, hydroxy propyl methyl cellulose, methyl cellulose, ethyl cellulose, and ethyl methyl cellulose, (ii) a polyacrylate or polymethacrylate; (iii) a gum, and (iv) a polyacrylamide, and (v) a polymer of polydiallyldimethylammonium chloride; (c) between 0.1% and 15% by weight of materials which promote microbial growth (microbial fertilizer) on and in the particle film and selected from slow release fertilizer particles including especially ammonium sulfate, phosphates, ureas, phosphonates, amino acids, and aspartic acid, e.g., polyaspartic acid.

In one embodiment, the particle film will additionally comprise an effective amount of a phthalocyanine dye, where the dye can help reduce heat stress of the plant and also reduce the undesirable white color of the particle film. Pigment green 7 and pigment blue 15 are preferred, and the amount can range from 0.05% to about 5%, for example 0.1% to 0.5% by weight, of the particle film. Small amounts of dye phthalocyanine particles have a large effect on the light transmission and reflectance from a particle film. Pigment green 7, copper phthalocyanine, substantially reduces the scatter properties of Surround due to its darker color.

In one embodiment, the particle film, when initially applied, may also contain a spreader, that is, a surfactant that causes the film to spread across a plant leaf surface. Spreaders, or spreading agents, are described in published US application 20070037711.

In one embodiment, the composition can further comprise one or more biologically active agents which can ameliorate oxidative damage, i.e., salicylic acid, kojic acid, azealic acid, and the like are advantageously present in amounts from about 1 ppm to about 100 ppm.

In one embodiment the particle film can be sprayed on the canopy of trees. While typical use of particle films is limited to high value crops, e.g., apples, pears, cherries, grapes, and certain fruits and vegetables, use on commodity crops such as on corn and use on trees, including broadleaf and pine forests, is also envisioned. In such cases, it may be beneficial to have the particle film be particularly rainfast by adding sticking agents and the like to the slurry.

Regarding the microbial fertilizer, advantageously the materials are packed for very slow release. If fertilizers are water soluble, it will more easily wash away or at least migrate to the low point of the leaf with daily wetting from dew. Additionally, fast release fertilizers will simply be washed out by rain or be absorbed by the plant foliage. Conversely, alfalfa or some similar organic source will be a physical part of the particle film, so that when the film is wetted, the structure of the particle film will hold the carbon source in place just as well as the kaolin. It should be noted that even routine applications of foliar fertilizers should encourage some microflora growth on particle films, but the amount and type of the microflora growth may not be ideal for ozone degradation or even for plant health.

In some applications fungicides and moldicides can be incorporated into the particle film matrix. It may be beneficial to include certain directed fungicides into the particle film, to reduce spread of undesired molds or fungi. Beneficial are lipopeptides, strobilurins, sulfur powder, and lime sulfur. Sulfur powder is long lasting and is readily incorporated into a particle film.

The goal is to stimulate microbial growth within the particle film and let the microbes increase the ozone degradation, but an environment that stimulates microbial growth can also stimulate disease. Emphasis is placed on providing particle films with nutrient profiles that promote bacteria, algae, bryophytes, and yeasts and not fungi since few plant pathogens are bacterial or yeast. The biofilm may advantageously contain fungicides. These include sulfur and lime sulfur particles, as well as strobilurins which have relatively low toxicity and have a broad range of horticultural crops they can be used on. Strobilurins are known for having excellent spectrum of control for pathogenic fungi and inducing a plant health benefit of their own. The biofilm may advantageously contain small amounts of ammonium sulfate and fertilizer grade micronutrients (termed microbial fertilizer, typically slightly soluble carbonates) to ‘fertilize’ the bacteria in the particle film. Calcium sulfate may also be useful, though only in small amounts. Use of compost teas to provide macro and micro nutrients to fertilize the biofilm in combination with a mineral particle film. Compost teas, which is essentially water washing of compost, can provide a source of ozone-destroying organics and other nutrients, and also provides inoculation of additional microbes as well as nutrients.

Some plant nutrients can be bound to the particle film if they are bound to the particles or to the dispersants. For example, polyaspartic acid is an excellent chelator and can therefore hold trace metal nutrients in the particle film, at least so long as it takes to deposit the film. Ascorbic acid and other vital nutrients can be anchored in the particle film by various methods, for example by forming an ester with a fatty acid that will adhere to particles. Other useful volumizing agents/spreader stickers include high molecular weight polyvinyl alcohol, especially partially hydrolyzed versions, and polyacrylamides. Polyacrylamides should be used sparingly as the promote water retention, which can benefit microflora in a particle film but which can promote disease if accumulations form.

The three dimensional aspects of a structured particle film can be enhanced by using larger particle sizes, e.g., 3 micron, and also by using a calcined kaolin particle source or different particle sources, e.g., kaolin and calcite, and by using dispersants that favor forming a structured particle film. The three dimensional particle film can protect beneficial microorganisms from uv degradation. Additionally, such a film may hold plant organic extrudates such as isoprene near the plant surface, where such extrudates can also react with ozone.

Bright white clay particle films such as Surround® and Purshade® reflect light, reduce canopy temperature, and increase photosynthesis. If ozone protection is the primary goal of the particle film, the film can be made less white or even be colored by for example phthalocyanine dye. A film can be formed of very small particles, e.g., 0.1 to 0.3 micron sized particles, which will be less visible but can still block or reflect some UV light, and can still form a framework to hold ozone-destroying organic materials. The film can even be formed of other materials, e.g., cellulose, activated carbon, small polymeric particles, or mixtures thereof with or without clays. Use finely ground organic matter (eg. Alfalfa, seaweed, and the like) can be used to form the particles (or fiber) in a particle film, to provide both the 3D matrix and additional nutrients with or without a mineral particle film to promote the development of the microbial film. The cellulose matrix will be slow to decompose so it may provide a 3D matrix over time. Such a film will also be less visible on plant surfaces. Again, use of slow release minerals e.g., various carbonates, to supply nutrients can be used with or without compost teas, and with or without an organic particle film.

Most commercial particle films are formed of a single component, and are typically highly visible. This is a function of both manufacturing efficiencies and a function of maximizing the other utilities of a particle film, that is, providing protection against sunburn and sun stress, lowering canopy temperatures, reducing arthropod infestations, and the like. Trees and crops treated with highly reflective particle films can reduce heat generation from the sun, which has environmental benefits. The coating is immediately visible to the naked eye, which does not deter most agricultural operations. In many uses, however, a bright white coating will not be desirable. Examples include treatment of trees in urban areas, treatment of ornamentals, and the like.

Adding minor amounts of copper phthalocyanine dye greatly reduced the white appearance of a Surround® particle film, though the combination of dye and particle film did not promote increased photosynthesis in plants as much as a bright white particle film. Basic optical properties of the Surround® particle film, a Pigment Green 7 particle film, and a 5 parts by weight Surround® and 1 part by weight of Pigment Green 7 are shown below. Measured values were transmission and reflectance of UV light (wavelength 280-320), near UV (wavelength 320-400), photosynthetic active light (wavelength 320-400), and IR light (wavelength 400-700).

| Deposition | | Transmission (%) | | | | Reflection (%) | | | |
|---|---|---|---|---|---|---|---|---|---|
| Material | g/m2 | UV | NUV | Vis | IR | UV | NUV | Vis | IR |
| G7 | 2.9 | 82 | 82 | 83 | 82 | 7 | 6 | 3 | 3 |
| Surround + G7 | 3.6 | 83 | 86 | 88 | 90 | 18 | 17 | 7 | 5 |
| Surround + G7 | 3.9 | 63 | 70 | 77 | 80 | 23 | 25 | 12 | 9 |
| Surround + G7 | 4.5 | 49 | 58 | 67 | 72 | 28 | 31 | 15 | 11 |
| Surround | 2.7 | 82 | 89 | 92 | 95 | 28 | 30 | 12 | 9 |
| Surround | 3.6 | 63 | 76 | 82 | 86 | 36 | 43 | 20 | 16 |
| Surround | 4.5 | 46 | 64 | 74 | 81 | 43 | 57 | 28 | 21 |

For unknown reasons, depositions of Surround®/dye compared with depositions of Surround® alone greatly reduced all light reflection and also reduced photosynthetically active light transmittance through the film, while increasing the UV light transmission through the film.

Treatments of Surround® without dye are known to increase plant photosynthesis and carbon fixation rates in a normal high-sunlight hot summer environment. A comparative study was done on plants treated with Green 7, with a film of Surround® and Green 7, with a film of Surround®, and with no particle film. Unfortunately, conditions were unfavorable for any particle film. The study was conducted from Jun. 2 to Aug. 1, 2011 and the data below reflect the change in canopy width, height, and weight. Plants were well watered and in a greenhouse that was kept ˜70-80 F during the day and had white wash on the greenhouse to limit heat but also limits light. Ozone levels were not elevated. Light levels in the greenhouse were about half of ambient. Therefore the plants were not light, heat or water stressed. The control is numerically the highest (most vigorous) because light is limiting Ps in the greenhouse and all the particle films reduce intercepted light. Indeed, reducing certain wavelengths of light and reducing temperature are the principal reasons growers use particle films. But in this test, light was scarce. Since Green 7 reduces light transmission through a particle film, we therefore expected the Surround®/Green 7 treatment to perform poorly. Plants treated with Surround®/dye at a 5:1 ratio did not grow as vigorously as plants treated with Surround® only. But the treatment was markedly less visible, and the dye is believed to be an effective ozone neutralizer. Under low stress conditions, there is no evidence of a photosynthetic beneficial effect of Green 7.

| | increase in | increase in | increase in |
|---|---|---|---|
| Treatment | width (cm) | height (cm) | weight (g) |
| Control | 40.4 | 33.1 | 1404 |
| Surround | 36.5 | 33.1 | 1279 |
| Surround + Green 7 | 30.2 | 26 | 1180 |
| Green 7 | 28.6 | 20 | 1145 |

Beneficially the particles and/or fibers forming the particle film can be primarily (e.g., greater than 50% by weight) cellulose or polymeric particles and/or fibers, where the particles and/or fibers have a diameter of for example between 0.1 and 50 microns, preferably between 1 and 10 microns, preferably between 1 and 10 microns, or between 2 and 15 microns. Cellulosic particle sizes can advantageously be larger than mineral particle sizes, and sizes above 2 mocons in at least one dimention can promote structure. In one embodiment an effective particle film can be formed from various materials where the film is substantially invisible. By substantially invisible we mean not readily apparent to an average person observing the treated plant from a distance of about 20 feet. A 25 to 500, for example a 200-500 microgram/square centimeter particle film formed primarily of cellulosic powder and/or fibers would be operable and substantially invisible. A 25 to 500, for example a 200-500 microgram/square centimeter particle film formed primarily of cellulosic powder and/or fibers and activated carbon would be operable and substantially invisible. A 25 to 500, for example a 200-500 microgram/square centimeter particle film formed from mixture of carbon and activated carbon powder would be substantially invisible. Films formed from mixtures of primarily cellulosic particles, but also having clay particles and/or calcite particles, will be barely visible, and may be substantially invisible if the mineral particles have a diameter less than about 0.3 microns. Low density films, e.g., less than 300 micrograms per square centimenter in density, formed from clay particles and/or calcite particles may be substantially invisible if the mineral particles have a diameter less than about 0.3 microns. It may be that inclusion of small amounts of cellulosic powder or <20 micron sized fibers may enhance the three dimensional aspects of the particle film, and the cellulosic powder would be a source, though a relatively inefficient source compared to a carbonaceous liquid dried on clay platelets, of carbonaceous material to react with ozone. And while a particle film made of hydrophobic particles, i.e., clay particles treated with fatty acid salts, are not desirable due to potential disease issues, a small amount, e.g., 0.5% to 20% by weight of hydrophobic particles will not form a watertrapping film that encourages disease but will provide a carbon source and will help fixate certain materials into the particle film.

In areas where the visibility of the treatment is not an issue, a white highly reflective particle film will under most summertime conditions result in increased plant growth and reduced arthropod infestations.

In field trials using enclosed canopies Surround® treated apple trees enhanced the degradation of ozone under field conditions. The particle film was originally envisioned to protect the individual treated leaves from the deleterious effects of elevated ozone levels. Enclosed canopy level testing revealed, however, that treated trees reduce the ozone levels in the entire canopy. That is, the treated vegetation becomes a filter that can reduce the ozone level in the ambient air. In preliminary studies, trees in enclosed chambers having controlled air throughput were treated with Surround®, and the ozone concentrations in and out of chambers were monitored. The Surround did not have any additional organics added thereto. Data is shown in FIG. 6. Data in dashed lines pertains to the y axis on the right, while the solid data pertains to the delta ozone (or change in ozone) y axis on the left. While ozone levels fluctuate greatly during the day, Surround-treated trees on average removed about 2 to 4 ppb of ozone more than untreated trees. The difference was most pronounced during the mid-day time period, when ambient ozone levels rose above 50 ppb. Very little difference was noted between the control (untreated trees) and the treated trees during the nights, when ambient ozone levels declined to about 20 ppb.

While the Surround®-treated trees reduced ozone more than untreated trees, the mechanism is not clear. It may be that the small amount of organics in the particle film and the high surface area of the particle film were responsible for the effect.

Regardless, a tree with a particle film, even a relatively ozone-inefficient particle film like Surround®, will reduce ambient ozone an appreciable amount. Further, reductions seem greatest when the ozone content is greatest. The amount is expected to be much greater when the particle films contain added organic material. This effect suggests that a sufficient density of treated plants and a large treatment area can reduce ambient ozone levels a significant amount, thereby benefitting untreated plants in the area. Therefore, having a large area of treated crops or trees of sufficient density can reduce ground-level ozone sufficiently to provide significant ozone-related damage amelioration even into untreated plants within the treated area. This effect depends on a large number of unrelated factors, e.g., wind and temperature, and the overall effect of removal of ozone from ambient air will of course only be significant where there is a sufficient density of treated plants.

In order to measure the true effect of ozone on apple trees, or other crops, controls must first be grown in conditions where the amount of ozone is known. Ambient ozone is dependent on a number of factors including temperature and even the time of the day (or night). Tests performed in growth chambers using carbon filtered air allows control, to for example a population of apple trees grown with ambient levels of ozone in WV (generally 30-40 ppb ozone). Each population will then be exposed to a range of ozone from 0, ambient, ambient+50 ppb ozone. These tests are ongoing.

All particle film liquid slurry applications which will form a particle film will require a volumizing agent to maximize the 3D component of the film. A spreader will ensure uniform particle film thickness. For those uses where the film is expected to persist through an entire growing season, a sticker will be added to resist rainfall erosion of the film

One embodiment of the invention is therefore a renewable ozone removing film that is constructed from a porous particle-based filter film architecture, located on plant surfaces where photosynthesis takes place, e.g., on leaves, where said film is supplied or activated with additions of nutrients and microbial inoculations. Another embodiment of the invention is a ozone removing film that is constructed from a porous particle-based filter film architecture, located on plant surfaces where photosynthesis takes place, where said particle film is supplied or activated with additions of a carbon source, such as alfalfa tea, which coats the particle surfaces. Another embodiment of the invention is a renewable ozone removing film that is constructed from a porous fiber-based filter film architecture, e.g., for example fibers of for example cellulose with a diameter of 0.1 to 20 microns, located on plant surfaces where photosynthesis takes place, e.g., on leaves, where said film is supplied or activated with additions of nutrients and microbial inoculations. The above embodiments can advantageously be combined. As discussed, the very thin layer of carbon on the very high surface area particle film can become exhausted in a matter of weeks, depending on how much organics were supplied and depending on the ozone levels. Existing carbon-based filters could be re-activated by the addition of nutrients and microbial inoculations, or with a spray of a carbon source such as compost tea or alfalfa tea, or both forms of renewal can be utilized. Existing fiber-based filters could be re-activated by the addition of nutrients and microbial inoculations.

While the effectiveness of an active-carbon coated particle film is confirmed, something else is needed. Alfalfa tea was just the starting point to determine if microbial growth could add to the ozone degradation process. We sprayed the ‘fermented’ organic material/Surround® materials on plants and measured an enormous increase in photosynthesis, much greater than plants treated only with Surround®. But the added effect was short-lived. Those carbonaceous material and microbes sacrificed their organics and cell membranes but rejuvenation is needed to keep the process going. This finding of ozone degradation has 2 commercial questions to address: 1) is there commercial value in protecting plants from ozone damage, 2) can a reliable and cost effective product be developed to meet this need?

It is possible to rejuvenate the organics in a particle film by re-applying solutions of organics at regular intervals, but that is not practical for most sites, and such spraying can easily promote disease.

The potential of a particle film reducing ozone damage leads to a possibility of using particle films on a number of crops not currently receiving particle film treatments. A cost effective ozone-related treatment need not be white, need not leave residues needing to be washed off of edibles before sale. A simple particle film of cellulosic particles and carboxyalkylcelluloses may form the bulk of an effective particle film, with perhaps a small amount (perhaps 0.1% to 10% by weight) of clays, calcium sulfate, calcium carbonate, or mixtures thereof to deliver desired minerals to the particle film. For many uses, the commercial angle is in the opposite direction of normal particle film technology—what's the minimum, most cost effective, and least visible and rainfast problem prone way to deliver ozone mitigation?

Certain volumizing agents useful for this invention have been previously disclosed. We believe the invention becomes useful when a particle film partially covers stomata, and has three dimensions (that is, more than a single layer of clay platelets), so that gases (including ozone) must diffuse through the particle film to reach the stomata. While spreader/stickers merely cause the film to spread across a greater percentage of a plant surface, volumizing agents cause on drying a three dimensional film to be formed. The volumizing agent acts as a cement, allowing the film to have a stable structure without the necessity of having particles jammed one against another. As a result, an ozone-directed particle film can be engineered to have greater permeability and porosity compared to a film of the same ingredients but without the volumizing agents. As gases diffuse through the porous particle film, ozone reacts with the various organic compounds present and becomes neutralized before reaching the stomata. The various organics which the ozone encounters include the surfactants and polymers used in the film (volumizing agents, spreader stickers, and the like), the ozone also encounters and is destroyed reacting with a microbe culture growing on and in the film, and/or by reacting with organics emitted from the plant and held in the film, e.g., isoprene.

The preferred films of this invention contain organics and microbes which can react with ozone passing through the film. The issue is to maintain a supply of organics and microbes. Organics can be applied with the film, and can also be exuded from the plant and be retained (even temporarily) in the particle film. Microflora/microbes are preferred as they can both repair ozone-induced damage and can regenerate. Most embodiments of this invention therefore contain nutrients for microbes. This includes both “micronutrients” like phosphate, sulfate, and ammonia, and can also include sources of carbon and/or nitrogen. Example include compost-tea, alfalfa tea, alfalfate particles, and the like. These are liquids separated after seeping with the compost, alfalfa, or other rich sources of polysaccarides, proteins, and microbes. Organic acids can provide sources of carbon and nitrogen. Various. fertilizers can provide other nutrients to the film. While foliar feeding with fertilizer is known, here the amount of nutrients is small and the solubility of the nutrients is controlled so that most micronutrients stay in the particle film as opposed to being absorbed by the leaves.

While foliar fertilization is well known in the art, the fertilizer particles here are very slow microbial nutrients designed and intended for very slow release within the film and the nutrients are substantially trapped in the particle film, thereby being useful to microbes growing in the film. Microbes will form and reform, maintaining the permeable carbon-nitrogen compound barrier between the stomates and the ozone-containing air, but still allowing permeation of gases (carbon dioxide and oxygen) necessary for photosynthesis.

A commercially available microbial product which can be incorporated as an adjuvant can be for example Actinovate AG, which is a high concentration of a patented beneficial bacterium on a water soluble powder. Actinovate AG contains the patented microorganism Streptomyces lydicus strain WYEC 108, which competes with and inhibits undesirable fungi while living at least partially feeding off of the plant's exudes while secreting beneficial and anti-fungal byproducts. These secretions can neutralize ozone diffusing through the particle film. This combination of the colonization and the protective secretions forms a defensive barrier around the plant which in turn suppresses/controls disease causing pathogens.

Advantageously, the particle film will additionally comprise an effective amount of a phthalocyanine dye, where the dye can help reduce heat stress of the plant and also reduce the undesirable white color of the particle film. The particle film itself will reflect some incident light, and also diffuse light so that undersides of other leaves can utilize the reflected light. But much of the harmful UV and IR radiation is blocked or adsorbed by the particle film. One disadvantage to this is the film has the appearance of a white or gray film can be un-appealing. Dyes, particularly phthalocyanine dyes, can be used to supplement the protective properties of the particle films. These dyes, when used in modest quantities, absorb harmful radiation but not photosynthetically useful radiation. While a number of phthalocyanine dyes are known, e.g., Pigment Blue 16, Vat Blue 29, Pigment Blue 15, Heliogen Green GG. Ingrain Blue 14, Ingrain Blue 5, Ingrain Blue 1, Pigment Green 37, and Pigment Green 7, the calcium-containing and copper-containing phthalocyanine dyes such as Pigment green 7 and pigment blue 15 are preferred. The amount is any amount that is visible. Too much phthalocyanine dye can be phytotoxic, but a small amount can further reduce heat stress in a plant, provide organics to react with ozone, and disguise the white color of the film. In our initial tests, the particle films contained about 17% by weight copper phthalocyanine. This amount is likely more than is needed to realize the appearance and antifungal effects of the dye. The amount of phthalocyanine dye in a particle film can range from 0.05% to about 15%, for example from about 0.05% to 5%, or for example 0.1% to 0.5% by weight, of the particle film.

Advantageously the particle film, when first applied to the plant surface as a water-born slurry, may also contain a spreader, that is, a surfactant that causes the film to spread across a plant leaf surface. The amount of spreader should be controlled, e.g., to between 0.01% to 1%, for example 0.05% to 0.4%, so the film can be effectively volumized.

Advantageously the composition can further comprise one or more biologically active agents which can ameliorate oxidative damage, i.e., salicylic acid, kojic acid, ascorbic acid, n-acetyl-L-cysteine, and the like are advantageously present in amounts from about 1 ppm to about 1000 ppm, more typically from 1 ppm to 100 ppm based on the weight of the dry particle film.

In one embodiment the particles in the particle film can comprise or consist essentially of kaolin and calcined kaolin. A commercial product is Surround® available from Tessenderlo Kerley Inc. Other useful particle sources are water-processed hydrous kaolin, silica free water-processed and degritted calcium carbonate, and water-processed montmorillonites. Smectite and bentonite can supplement the particle film and also stabilize the slurry during deposition of the film.

FIG. 5 shows results of a study measuring the effect of a particle film on the degradation of ozone. As you will see, the particle film, that is, Surround®, in the presence of organic molecules (nutrient broth “NB” and bacteria “B”) will degrade ozone—completely. The test tubes were packed with steel balls. In controls, NB and B were added to test tubes without the particle film. The broths were dried before testing. The organics alone had little effect on the ozone passing through the tube. The presence of the particle film, with the NB, B, or both, resulted in sharp dramatic drops in ozone exiting the tubes. Without being bound by theory, we expect the film provides the surface area/porosity available for contact with the ozone. If these treatments are left in contact with a continual source of ozone for extended periods, the ozone will eventually degrade all the organic matter and the values will return to ambient. The study was done by filling each tube with the corresponding solution and drying it for 3 days at 60 C. The drying will killed any bacteria but does not oxidize the organic matter. The ozone generator created an air supply with 240 ppb ozone (a reasonable air pollution level) and this air stream was directed into the bottom of each tube with a glass tube (Pasteur pipette). The ozone then diffused up to the top of the tube where it was measured. The air flow into the tubes was 83 volume exchanges per minute—a very fast and unnatural rate that really challenged the system.

The results were so dramatic that we hypothesized that some trees treated with a particle film might be able to substantially affect the amount of ozone in the ambient air. This was tested and found to be the case. FIG. 6 shows the reduction of ozone in ambient air passing through a growth chamber could be significantly reduced. Additionally, the plant itself will be protected from the damaging effects of ozone.

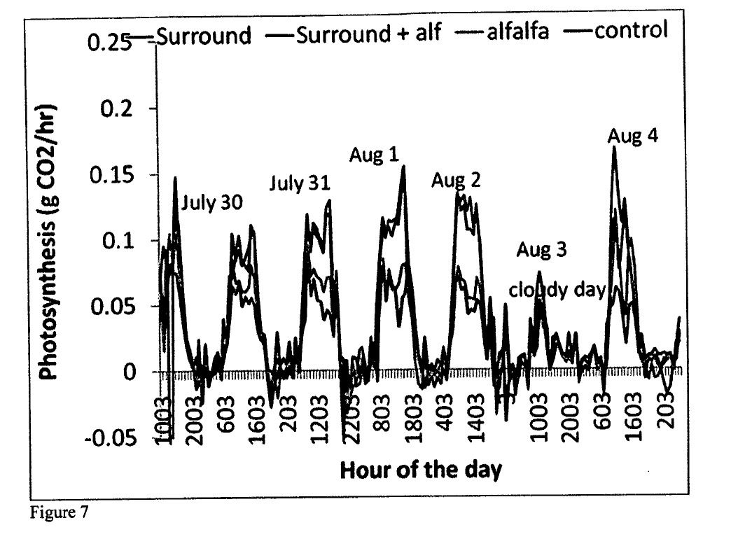

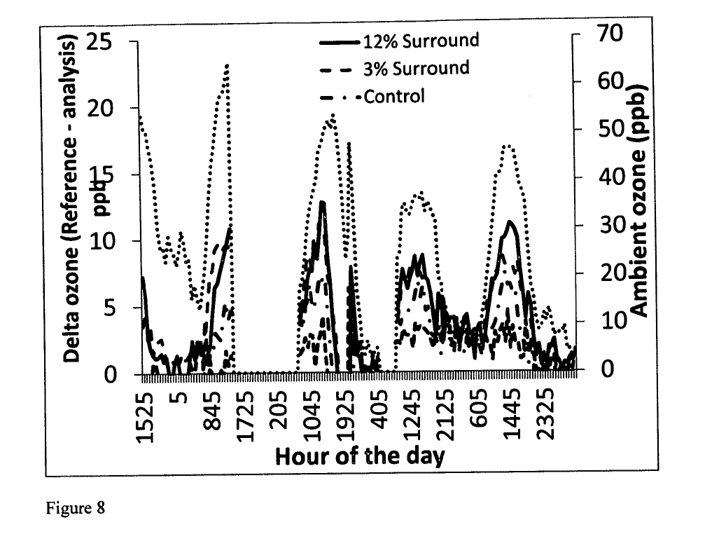

Additional field trials were conducted where plants in the chambers were exposed to elevated ozone (about 100 ppb above ambient). In these tests, the plants were expected to be substantially stressed by the ozone. Therefore, photostynthesis rates based on carbon assimilation were being monitored. There were four tests: 1) a control 1 (no particle film, no alfalfa, 2) alfalfa dust sprayed on as a slurry, 3) Surround and 4) Surround+alfalfa dust sprayed on as a slurry. The growth rate data is summarized in FIG. 7. Every 2-3 days treatments were re-applied as the plants grew, to treat new leaves. The photosynthetic rates of the plants treated with Surround® and with Surround® plus alfalfa were 50% to 100% greater than the photosynthetic rates of the control plants. Surprisingly, spraying with alfalfa dust provided only a small improvement in photosynthesis as no treatment. A first surprising result was therefore the marginal effect of alfalfa dust (estimated particle size between 3 and 10 microns) alone, at least before the fermented alfalfa slurry was sprayed. The presence of a porous permeable clay structure, with very high resulting surface areas, is therefore important in achieving best results. The increase also was not simply a fertilizer effect—the treatment with only alfalfa was only marginally better than the control samples. It seems the combination of alfalfa dust and Surround together provided the benefit. An alfalfa dust (tea) treatment that had fermented for 3 days, and which contained obvious microbial content, was applied on August 4. Both the alfalfa alone and the Surround+alfalfa treatments made large increases in photosynthesis compared to their previous photosynthesis rates. While on some days the Surround plus alfalfa did not seem to contribute a large amount of increased photosynthesis by itself, when the alfalfa tea dust was sprayed with Surround results went up dramatically. So it appears that the microbial component (in the alfalfa tea) can add to the degradation of ozone in a very significant manner. Alternatively or additionally, organic material leached from the alfalfa dust during three days of soaking coated the high surface area Surround film, thereby strongly reducing the ozone content of the gas affecting the plant and thereby more than doubling the photosynthesis rate compared to the control.

Plants exude nutrients and carbon compounds to their surface. These exudates support a vast ecosystem of fungi, bacteria and yeasts. The particle film allows these exudates to diffuse into a more 3-D configuration with greater surface area which supports these microbial populations in addition to collecting organic dust that floats into the plant leaf. The live and dead bodies of the microbes are likely the agents that react with the ozone to convert it to water and oxygen. What drives this system to work and degrade ozone is the biological activity that develops in the film; both the living organisms but more importantly the dead cells. The biological question is whether the plant-film-microbe complex regenerates sufficient degradation sites each day to handle the ozone load. Photosynthetic microbes can also be useful.

Advantageously the film is between 0.1 and 10 microns thick, more typically 0.5 to 3 microns in thickness. Such a film could readily degrade 30 ppb of ozone diffusing therethrough.

Advantageously the film has less than 0.25%, preferably less than 0.1% or less than 0.5% of crystalline silica.

In one embodiment the invention is an enhanced biofilm comprising a three dimensional network of particles, e.g., 0.1 microns to 5 microns, typically 0.2 microns to 1 micron average particle diameter. The particle film is treated to promote the accumulation and retention of organics to further enhance ozone degradation. In one embodiment ammonium sulfate and fertilizer grade micronutrients (termed microbial fertilizer) are added to the film to ‘fertilize’ the bacteria in the particle film. Use of standard foliar fertilizer agents (eg. ammonium sulfate, urea, calcium nitrate, micronutrient sprays) to stimulate the microbial film is also contemplated.

Incorporation of amino acids into the particle film as C and N source to stimulate the microbial film in addition to mineral film/plant film is useful.

All particle film slurries will benefit from effective amounts of a volumizing agent to maximize the 3D component of the film, a spreader used to ensure uniform particle film thickness, and a sticker to resist rainfall erosion of the film. This is especially important when treating for example mature trees and even evergreen trees.

A renewable ozone filter can be constructed from a porous mineral-based filter re-activated with additions of nutrients and microbial inoculations. Existing carbon-based filters could be re-activated by the addition of nutrients and microbial inoculations. Existing fiber-based filters could be re-activated by the addition of nutrients and microbial inoculations. Ozone flux through these trees will be measured on large apple trees in the field. The initial laboratory study demonstrated that total ozone degradation could be accomplished with a Surround-biofilm.

One factor is the retention of the treatment on the plant surface for a time sufficient to achieve the desired result. In this connection, adequate retention times indicate that properties such as resistance to time, wind, water, mechanical or chemical action are possessed. Another factor is proper coverage of the treatment to provide appropriate coverage over the plant surface. Proper coverage may involve modifying the surface tension of spray droplets, increasing surface wetting, and/or enhancing coverage. Another factor is the nature of the deposition itself, which needs to be appropriate to maximize the effect of the application. It is difficult to provide topical agricultural or horticultural treatments with desirable retention characteristics, desired deposition, and proper coverage. For example, often, improving retention characteristics results in reducing proper coverage, and vice versa. In another example, improving coverage can have undesirable deposition characteristics. A key strategy in applying to plants is the consideration of the hydrophobic to hydrophilic nature of plant surfaces. Also, substrate characteristics such as orientation, form, purity, texture, and rigidity are to be considered.

Applications of liquids to hydrophobic surfaces are problematic as these surfaces repel aqueous-based sprays. This is usually remedied by use of a surfactant. However, depositions with surfactants used by themselves can be too thin and can run off hydrophobic surfaces and, in addition, can be extremely thin and have extreme run off of co-targeted hydrophilic surfaces. Thus, in terms of hydrophilic surfaces, conventional agricultural surfactants (spreaders) used by themselves can overspread and cause extreme runoff resulting in poor coverage.

There are two techniques currently used to improve delivery of particles to target surfaces. One is the retention of the treatment on the plant surface by the use of stickers. The second factor is the use of spreaders to improve coverage of the treatment. These arts can enhance spray retention on hydrophobic surfaces but overspreading and droplet retraction occurs which leads to the problem of thin, spotty deposits and/or non-uniform film formation. When spreaders are used in hydrophilic surfaces run off is a problem. There is also a need for spreading and sticking agents that have relatively equal deposition properties on both hydrophobic and hydrophilic surfaces. This is particularly needed in plants that have both hydrophobic and hydrophilic surfaces such as tomatoes and grapes wherein generally the fruit is hydrophobic and the foliage is hydrophilic. In such a case, a given level of conventional spreaders may be ideal for the hydrophobic part of the plant, but may induce overspreading on the hydrophilic part of the plant.

Prior art particle films are used for sunburn and heat stress reduction and rely on the light properties passing through the particle film, in particular the controlled blockage of visible, UV, and IR light, to gain beneficial effects. Improved particle film treatments with improved controlled blockage of light and film-forming spreading (defined below) for both hydrophilic and hydrophobic agricultural substrates are therefore desired. Optical properties are beneficial for an ozone-directed particle film, but are not essential.

The present composition is capable of forming a particle film and comprises: (a) between 50% and 99% by weight of at least one particle; (b) at least one volumizing agent which optionally can be selected from the group consisting of: (i) cellulose selected from the group consisting of ethyl hydroxy ethyl cellulose, hydroxy ethyl cellulose, hydroxy propyl cellulose, hydroxy ethyl methyl cellulose, hydroxy propyl methyl cellulose, methyl cellulose, ethyl cellulose, and ethyl methyl cellulose and present in an amount greater than 0.35% by weight; and (ii) non-cellulosic component or cellulose other than said cellulose (i) present in an amount of at least 0.05% by weight; and optionally (c) at least one spreader.

In one example, the present composition comprises: (a) particles, and (b) gelatin. Gelatin is a useful volumizing agent and is a ready source of C and N for microflora. The volumizing agents of (b) do not, per se, have the ability to spread on hydrophobic surfaces. The present composition forms volumized films when wet or dry. At least one of the following may also be present: a conventional agricultural spreader, polymeric film-forming agent, agricultural sticker, functional additive, or facilitator.

Volumized compositions maximize the height of the deposition and increase friability of the particle film. A main benefit of volumization of prior art particle films is the increase in opacity known to occur via the phenomena of scattering of light due to flocking or flocculation of the particles. It is known that if air interfaces are created between particles much like a house of cards, light scattering, and therefore opacity, is increased. This phenomena is seen in such substances as snow (versus ice) and crushed glass (versus uncrushed glass). In ozone-directed films, the important factors are film thickness, permeability, and availability of reactive carbon sources. Using volumization agents, hydrous kaolin particle film compositions can be prepared that have permeability and porosities as good as particle film compositions using the more expensive calcined kaolins.

Certain volumization agents act as an effective spreading inhibitor. The phrase “spreading inhibitor” as used herein means a substance that has both low spreading on hydrophobic surfaces and may prevent other known spreaders from spreading. Examples of spreading inhibitors include low molecular weight hydroxylethyl cellulose (HEC) and carboxymethyl cellulose (CMC). In this way, novel depositions, for example, can be attained with compositions that do not spread on hydrophobic surfaces thus forming purposely discontinuous or spotty coverage that can be advantageous for enhanced insect repellency.

Further novel compositions can be made with volumizing agents and spreading agents to achieve film-forming spreading on hydrophobic surfaces that is similar to the film-forming spreading achieved on hydrophilic surfaces (including a co-sprayed hydrophilic surface).

The term “structure” or “structuring” as used herein means having the ability to cause individual particles to form flocks, agglomerates, aggregates, and/or associations that can cause a system to be volumized upon drying and thereby constructs a functional deposition.

The term “volumized” as used herein means the increased separation of a given mass of particles. Volumized usually results from structuring as defined above or may also result from increasing viscosity and/or surface tension. In most cases, this means that the resultant dried deposition, wet deposition or wet sediment has a greater volume than the same deposition that is not volumized. Volumized also means that depositions are higher and thicker in the liquid state (before drying).

The phrase “volumizing agent” as used herein means any agent capable of constructing a volumized system that does not spread, per se, on hydrophobic surfaces, but spreads readily on hydrophilic surfaces.

The term “sticker” as used herein means a material that increases the adhesion of sprays on plants by resisting various environmental factors. Sticker may also increase the firmness of attachment of spray emulsions, active ingredients, water soluble materials, liquid chemicals, finely-divided solids or other water-soluble or water-insoluble materials to a solid surface, and which may be measured in terms of resistance to time, wind, water, mechanical or chemical action. A sticker may be further defined as a material which increases spray droplet retention to a substrate by facilitating droplet capture and thereby preventing the material from rolling off, blowing off, deflecting, shattering, or otherwise reducing the amount of spray material which remains in contact on the substrate during moment of deposition until the time which the spray droplet has chance to dry.

The phrase “particle film” as used herein means a film composed substantially of particles.

The term “film forming spreading” as used herein means a type of spreading that also builds films having increased fluid volume retention and thus increased solids deposition on similarly both hydrophilic and hydrophobic surfaces.

A volumized particle film results in a higher level of efficiency per number of particles per a given mass of film. Due to the volumized and/or flocked or otherwise associated structure, several advantages are obtainable. The volumized particle film has highly separated particles. The volumized film exhibits improved elastic properties, flexural properties and energy buffering properties making it less vulnerable to cracking, chipping, an/or flaking, thereby improving weatherability by reducing wash-off and wind attrition while improving adhesion. The volumized particle film is less likely than a conventional spread film to have its particles deeply embedded in the waxy cuticle of fruit. When employing particles on plants, the volumized particle film improves scattering of undesirable or excessive infrared, visible, and ultraviolet light. Also, because more uniform depositions are produced, more uniform light is transmitted to the substrate resulting in more uniform color and less mottling. The volumized particle film has improved insect control compared to a conventional spread film due to its increased friability, greater surface area and greater number and mass of particles available to contact the pest.

Examples of such volumizing agents include glues, gelatins, collagens, hydrolyzed collagens, magnesium aluminum silicates, colloidal clays, cellulose polymers, polyacrylates, polyacrylamide (PAM), polyamines (epichlorohydrin-dimethylamine); polydiallyldimethylammonium chloride (polyDADMAC), epichlorohydrin-dimethylamine (Epi-DMA), and gums such as locust bean gum, xanthan gum, guar gum, carrageenan, and Psyllium.

Glues are generally considered to be adhesives consisting of organic colloids of a complex protein structure obtained from animal materials such as bones and hides in meat packing and tanning industries. Glues generally contain two groups of proteins: namely, chondrin and glutin. Gelatin is one of the main constituents of animal glue. Gelatin materials include gelatin, collagen, and glue and are commercially available from a number of sources. While not wishing to be bound by any theory, it is believed that the gelatin materials facilitate the formation of particulate material agglomerates as well as facilitate binding between particulate material agglomerates and substrates.

Particle films can comprise magnesium aluminum silicates or colloidal clays including attapulgites or bentonites. Attapulgites and bentonites may be beneficiated or otherwise processed.

Cellulose polymers are complex carbohydrates (polysaccharides) of thousands of glucose units in a generally linear chain structure. Celluloses are generally water-soluble polymers. Celluloses include one or more of non-hydrolyzed, partially hydrolyzed, substantially hydrolyzed, and fully hydrolyzed celluloses. Examples of celluloses specifically include ethyl hydroxy ethyl cellulose, hydroxy ethyl cellulose, hydroxy propyl cellulose, hydroxy ethyl methyl cellulose, hydroxy propyl methyl cellulose, methyl cellulose, carboxy methyl cellulose, sodium carboxy methyl cellulose, ethyl cellulose, ethyl methyl cellulose, cross-linked sodium carboxymethyl cellulose, enzymically hydrolyzed carboxymethylcellulose, and the like. Celluloses are commercially available from numerous sources. Cellulose volumizing agents have the ability to create a purposely discontinuous or spotted film on surfaces. This trait is useful in creating spotted particle films deposition patterns that can disguise fruit or crops from insects such as fruit flies, thus lowering insect damage. Examples of cellulose types that form spots on hydrophobic surfaces are hydroxylethyl cellulose, carboxy methyl cellulose, sodium carboxy methyl cellulose, cross-linked sodium carboxymethyl cellulose, enzymically hydrolyzed carboxymethylcellulose, and the like. Other examples include polyacrylates having molecular weight of 250 to about 10,000, polymethylacrylate, polyethylacrylate, polyacrylic acid, polymethylmethacrylate, polyethylmethacrylate, poly (2-hydroxyethyl methacrylate), and high molecular weight polyacrylamides.

In addition, finely divided, low density (<1.0 g/m) insoluble materials, materials minimally or partially soluble, or materials from the above group which are minimally soluble may function as volumizing agents via buoyancy and density differences. Examples include high molecular weight (>85000) polyvinyl alcohols, cross-linked polyvinyl alcohols, fully hydrolyzed polyvinyl alcohols, micronized thermoplastics, and powdered waxes.

The present composition may additionally comprise a conventional agricultural spreader that causes the volumized composition to attain film-forming spreading similarly effectively on both hydrophobic and hydrophilic surfaces. Such products can increase spreading and thus coverage area of volumized compositions that normally resist spreading on incompatible surfaces (usually hydrophobic). These spreaders are composed of a surfactant or surfactants and other ingredients that improve film-formation. Conventional spreaders are nonionic, anionic, cationic, or amphoteric. Examples include modified phthalic glycerol alkyd resins such as Rohm & Haas' Latron B-1956, plant oils such as cotton seed oil or cocodithalymide such as Sea-wet from Salsbury Lab, polymeric terpenes such as Pinene II from Drexel Chem., and ethoxylated tall oil fatty acids such as Toximul 859 and Ninex MT-600 from Stepan. Other useful spreaders include nonionics such as alkyl polyglucosides and octylphenol ethyoxylates, and anionics such as dioctyl sulfosuccinates, phosphate esters, sulfates, or sulfonates such as Dow's Triton™ products. Other useful spreaders include nonionics such as branched secondary alcohol ethoxylates, ethylene oxide/propylene oxide copolymers, nonylphenol ethoxylates, and secondary alcohol ethoxylates such as Dow's Tergitol™ products. Other useful spreaders include organosilicones such as Silwet and phenoxyethanol such as Igepal.

The base particles used in the particle film can be hydrophobic or hydrophilic. Hydrophillic particles are typically preferred. The particles can be hydrophobic in and of themselves, (for example, mineral talc). Alternatively, the particles can be hydrophilic materials that are rendered hydrophobic by application of a surface treatment such as a hydrophobic wetting or coupling agent; for example, the particle has a hydrophilic core and a hydrophobic outer surface. In another alternative embodiment, the particles are hydrophilic in and of themselves, for example calcined kaolins. In yet another embodiment, the particles are hydrophobic in and of themselves and made hydrophilic by the addition of wetting agents such as surfactants or emulsifiers. Examples of base particles suitable for use in the present invention includes processed minerals, such as water processed kaolin; air processed kaolin; hydrous kaolin; calcined kaolin; anhydrite; sillimanite group minerals such as andalusites, kyanites, sillimanites; staurolite, tripoli; tremolite; gypsum (natural and synthetic); anhydrite; adobe materials; barites; bauxite or synthetic aluminum trihydrate; fine aggregated material less than 50 microns median particle size diameter, both lightweight and dense such as crushed or milled stones, gravels, silicas, silica flours, pumices, volcanic cinders, slags, scorias, expanded shales, volcanic cinders, limestones such as calcites and dolomites; diamond dusts both synthetic and natural; emerys; biotites; garnets; gilsonites; glauconites; vermiculites, fly ashes, grogs (broken or crushed brick), shells (oyster, coquina, etc.); wash plant or mill tailings, phosphate rocks; potash; nepheline syenites, beryllium materials such as beryls; borons and borates, calcium carbonates both ground and precipitated, talcs, clay minerals such as fullers earths, ball clays, halloysites, refractory clays, flint clays, shales, fire clays, ceramic clays, coal containing kaolins, bentonites, smectites (montmorillonite, saponites, hectorites, etc); hormites (attapulgites, pyrophyllites, sepeolites, etc.); olivines; feldspars; sands; quartz; chalks; diatomaceous earths; insulation materials such as calcium silicates, glass fibers, mineral wools or rock wools; wollastonites; graphites; muscovites; micas; refractory materials; vermiculites; perlites; glass fibers; rare earth minerals; elemental sulfurs and other sulfur minerals; other insoluble elemental and salt compounds; other miscellaneous insoluble particles; other functional fillers such as, pyrogenic silicas, titanium minerals such as titanium dioxides, magnesium oxides, and magnesite.

Typically various forms of calcite, various forms of kaolin, bentonite, montmorillonite, and attapulgite are preferred mineral particles. Zeolites, diatomaceous earth, and amorphous silica are also contemplated. If the term the term “calcites” as used here includes calcium carbonates, calcium magnesium carbonates, and even primarily magesium carbonates (magnesite), which typically but not always contains some calcium. Typical natural calcites are mixed crystals that contain 80 to 99% by weight calcium carbonate and 1 to 20% by weight magnesium carbonate.

Examples of non-mineral base hydrophilic particles include carbon soot, coal dust, ash waste and other colored organic materials. Organic materials such as cellulose fibers; wood fiber; vegetable fibers such as bamboo, hemp, jute, sisal and the like; synthetic fibers such as nylon, aramid, polyethylene, polytetrafluoroethylene; animal fibers such as wool, etc. The particles must be very small, e.g., less than 50 microns in any diameter, to facilitate ease of manufacturing, handling, and spraying. Another example of a functional additive is dark pigments.

All materials may be considered useful to this invention whether incorporated in their natural/crude/hydrous form, in processed forms including water washing, air floated, beneficiated, and synthetically produced. Further processing can include heat treatment above 400 degrees Fahrenheit, more commonly referred to as calcination.

Heat treatment in accordance with the invention commonly involves heating a particle at a temperature from about 100.degree. C. to about 1,200.degree. C. for about 10 seconds to about 24 hours. In another embodiment, heat treatment involves heating a particle at a temperature from about 400.degree. C. to about 1,100.degree. C. for about 1 minute to about 15 hours. Heat-treated particles are generally hydrophilic. Specific examples include metakaolin, calcined calcium carbonate, calcined talc, calcined kaolin, baked kaolin, fired kaolin, hydrophobic treated heat treated kaolin, calcined bentonites, calcined attapulgite, calcined clays, calcined pyrophyllite, calcined feldspar, calcined chalk, calcined limestone, calcined precipitated calcium carbonate, calcined diatomaceous earth, calcined barytes, calcined aluminum trihydrate, calcined pyrogenic silica, and calcined titanium dioxide. Heat treating cellulosic particles is best performed at lower temperatures, for example between about 120 degrees C. to 200 degrees C. and can be done in the an oxygen-deficient environment.

The particles suitable for use in the present invention are finely divided. The term finely divided when utilized herein means that the particles have a median individual particle size (average diameter) below about 100 micrometers. In one embodiment, the particles have a median individual particle size of about 10.micronsor less. In another embodiment, the particles have a median individual particle size of about 3 microns or less. In yet another embodiment, the particles have a median individual particle size of about 1 micron or less. Particle size and particle size distribution of mineral particles as used herein are measured with a Micromeritics Sedigraph 5100 Particle Size Analyzer. Measurements are recorded in deionized water for hydrophilic particles. Typically, for kaolin 0.5% tetrasodium pyrophosphate is used as a dispersant; with calcium carbonate 1.0% Calgon T is used. Typical densities for the various powders are programmed into the sedigraph, for example, 2.58 g/ml for kaolin. The sample cells are filled with the sample slurries and the X-rays are recorded and converted to particle size distribution curves by the Stokes equation. The median particle size is determined at the 50% level.

The present invention may also include other functional additives. One example of a functional additive is cross-linking agents. Cross-linking agents, when combined with cross-linkable polymers, facilitates the formation of a volumized system. The cross-linking agent reacts with the cross-linkable polymers to increase the molecular weight. Examples of cross-linking agents include borax, glyoxal, alkylene glycol methacrylates, ureaformaldehyde, polyamines, and the like. As an example of a cross-linked polymer, a high molecular weight polyvinyl alcohol may be cross-linked with borax or polyacrylamide may be cross-linked with ethylene glycol dimethacrylate.

The volumized particle film may additionally be used for pest/insect control, disease control, pesticide delivery systems, solar protection/reducing sunburn, ground-applied light reflectants, heat stress reduction, preventing damage from freezing temperatures, weed control, reducing physiological disorders such as watercore, corking and bitterpit, increasing the resistance to freeze dehydration, and the like.

Plant surfaces include those found on crops, household and ornamental plants, greenhouses, forests with types of surfaces that include leaves or needles, stems, roots, trunks, or fruits, and include soil or other growth mediums, and the like. The substrates on which the volumized film may be formed can include horticultural crops such as actively growing agricultural crops, fruiting agricultural crops, actively growing ornamental crops, fruiting ornamental crops and the products thereof, and surfaces pests infest such as man-made structures, soil, and stored grains/fruits/nuts/seeds, as well as the surfaces of pests. Specific examples include fruits, vegetables, trees, flowers, grasses, and landscape plants and ornamental plants. Specific examples of plants include apple trees, pear trees, peach trees, plum trees, lemon trees, grapefruit trees, avocado trees, orange trees, apricot trees, walnut trees, raspberry plants, strawberry plants, blueberry plants, blackberry plants, boysenberry plants, corn, beans including soybeans, squash, tobacco, roses, violets, tulips, tomato plants, grape vines, pepper plants, wheat, barley, oats, rye, triticale, and hops.

The slurry is applied to the target surfaces by spraying, or other suitable means. The particle treatment may be applied as one or more layers. The amount of material applied varies depending upon a number of factors, such as the identity of the substrate, the purpose of the application, and the identity of the particle, etc. In any given instance, the amount of material applied can be determined by one of ordinary skill in the art. The amount may be sufficient to form a continuous film or intermittent film over all or a portion of the substrate to which the particle treatment is applied. One or more layers of this dust, slurry, cream or foam may be dusted, sprinkled, sprayed, foamed, brushed on or otherwise applied to the surface. The resultant particle film residue, whether formed by a dry or slurry application, may result in coatings that are hydrophilic or hydrophobic.

The present agricultural compositions may be used to enhance photosynthesis as disclosed in U.S. Pat. No. 6,110,867, incorporated in its entirety herein by reference. In an embodiment, the thickness of the particle film ranges from about 3 microns to about 3,000 microns. In yet another embodiment, the thickness of the particle film ranges from about 5 microns to about 750 microns. The present agricultural composition may be applied from about 25 up to about 5,000 micrograms of particle per cm2 of surface for particles having specific density of around 2-3 g/cm³, more typically from about 100 up to about 3,000, and preferably from about 100 up to about 500 micrograms of particle per cm2 of surface. In addition, environmental conditions may reduce coverage of the particle film and multiple applications may be desirable.

In one embodiment, the volumized films made in accordance with the present invention do not materially affect the exchange of gases (other than ozone) on the target surface. The gases that pass through the particle treatment (or residue from the inventive treatment) are those that are typically exchanged through the target surface and the environment Such gases, vapors or scents include water vapor, carbon dioxide, oxygen, nitrogen, volatile organics, fumigants, pheromones and the like.

The Examples and tests are exemplary rather than exhaustive and are so intended.

1. A method of protecting plants from ozone comprising: applying to the plants a particle film containing
A) between about 50% and 99.4%, for example between 70% and 90% by weight of particles selected from the group consisting of mineral particles, polymeric particles and/or fibers, cellulosic powder and/or fibers, and charcoaled (activated) carbon particles;
B) at least one volumizing agent in an effective amount;
C) two or more of:
c1: between 0.1% and 25% by weight, for example between 5% and 20% of active nitrogen-rich carbonaceous materials which destroy ozone, said materials being immobilized in the particle film and in preferred embodiments comprising one or more of polyamines, poly-amino acid derivatives;
c2: between 0.1% and 25% by weight, for example between 5% and 15% by weight of materials which promote microbial growth (microbial fertilizer) on and in the particle film and selected from slow release fertilizer particles and microflora nutrients including primarily sources of C and N; and
c3: between 0.1% and 25% by weight for example between 5% and 20% by weight of active carbonaceous materials coated on the particles, said carbonaceous materials comprising ozone-reactable carbon sources, for example organic teas, such as alfalfa teas, compost teas, fermented organic solutions, and the like, and
D) optionally one or more of:
0.01% to 10%, for example 0.1% to 5%, of beneficial bacteria or microflora;
fatty acid esters of ascorbic acid;
0.1% to 10% of a spreader/surfactant that causes the film to spread across a plant leaf surface; effective amounts of biologically active agents which can ameliorate oxidative damage, i.e., ascorbic acid, azealic acid, salicylic acid, kojic acid, and the like, for example present in amounts from about 1 ppm to about 100 ppm; and
0.01% to 20% of a phthalocyanine dye, for example pigment green 7;
said particle film having a dry weight of between 25 and 5000 micrograms per square centimeter.

2. The method of protecting plants from ozone of claim 1 wherein the particles in the particle film comprises primarily mineral particles selected from calcium carbonates, kaolins, attapulgite, montmorillonites, bentonite, and/or calcined kaolins.

3. The method of protecting plants from ozone of claim 1 wherein the particles in the particle film comprises primarily cellulosic particles and/or fibers.

4. The method of protecting plants from ozone of claim 1 wherein the particles in the particle film comprises primarily polymeric particles and/or fibers.

5. The method of protecting plants from ozone of claim 1 wherein the particles in the particle film comprises a mixture of cellulosic particles or fibers and mineral particles.

6. The method of protecting plants from ozone of claim 1 wherein the particle film has a density between greater than 100 micrograms per square centimeter and the particle film is substantially invisible.

7. The method of protecting plants from ozone of claim 1 wherein the particle film has a density between greater than 100 micrograms per square centimeter and the particle film is substantially invisible.

8. The method of protecting plants from ozone of claim 1 wherein the particle film comprises between 5% and 20% by weight of ozone-reactable polyamine carbonaceous material coated on the particles, and between 5% and 15% by weight of microbial fertilizer.

9. The method of protecting plants from ozone of claim 1 wherein the particle film comprises between 5% and 15% by weight of microbial fertilizer on and in the particle film, and between 5% and 20% by weight of ozone-reactable carbonaceous materials coated on the particles.

10. The method of protecting plants from ozone of claim 1 wherein the particle film comprises between 5% and 20% by weight of ozone-reactable carbonaceous material coated on the particles, and between 5% and 20% by weight of ozone-reactable carbonaceous materials coated on the particles.

11. The method of protecting plants from ozone of claim 1 wherein the particle film comprises 0.01% to 10%, of beneficial bacteria or microflora selected from Streptomyces, Bacillus sp., and bryophytes.

12. The method of protecting plants from ozone of claim 1 wherein the volumizing agent is selected from modified celluloses and high average molecular weight polyvinyl alcohol of molecular weight greater than 85000.

13. The method of protecting plants from ozone of claim 1 wherein the particle film comprises polyaspartic acid, poly-amino acids, or mixtures thereof.

14. The method of protecting plants from ozone of claim 1 wherein the method further comprises subsequent applications of nitrogen-rich carbonaceous materials which destroy ozone, microbial fertilizer, or active carbonaceous materials.

15. The method of protecting plants from ozone of claim 1 wherein the particle film comprises fatty acid esters of ascorbic acid.

16. The method of protecting plants from ozone of claim 1 wherein the particle film comprises cellulose or polymeric particles and/or fibers, where the cellulose or polymeric particles and/or fibers have a diameter of between 0.1 and 50 microns.

17. The method of protecting plants from ozone of claim 1 wherein the cellulose particle film comprises organic teas, alfalfa powder, glucose, sucrose, corn starch, apple pumice, or casein, dried onto the particles.

18. A method of protecting plants from ozone comprising: applying to the plants a particle film containing
A) between about 50% and 90% by weight of particles selected from the group consisting of mineral particles, polymeric particles and/or fibers, cellulosic powder and/or fibers, and charcoaled (activated) carbon particles;
B) at least one volumizing agent in an effective amount;
C) two or more of:
c1: between 0.1% and 25% by weight, for example between 5% and 20% of active nitrogen-rich carbonaceous materials which destroy ozone, said materials being immobilized in the particle film and in preferred embodiments comprising one or more of polyamines, poly-amino acid derivatives;
c2: between 0.1% and 25% by weight of materials which promote microbial growth (microbial fertilizer) on and in the particle film and selected from slow release fertilizer particles and microflora nutrients including primarily sources of C and N; and
c3: between 0.1% and 25% by weight for example between 5% and 20% by weight of active carbonaceous materials coated on the particles, said carbonaceous materials comprising ozone-reactable carbon sources, for example organic teas, such as alfalfa teas, compost teas, fermented organic solutions, and the like,
wherein the minimum amount of carbonaceous material in the particle film, excluding the particles, is at least 15% by weight, and
wherein the particle film results in amelioration of ozone-related photosynthesis reduction by an amount equivalent to a reduction of daytime levels of ozone of at least 10 ppb.

19. The method of protecting plants from ozone of claim 18 wherein the minimum amount of carbonaceous material in the particle film, excluding the particles, is at least 20% by weight, and wherein the particle film results in amelioration of ozone-related photosynthesis reduction by an amount equivalent to a reduction of daytime levels of ozone of at least 20 ppb.

20. The method of protecting plants from ozone of claim 18 wherein the amelioration of ozone-related photosynthesis reduction by an amount equivalent to a reduction of daytime levels of ozone of at least 40 ppb.
